SEQUENCE LISTING (3 sequences)

SEQ ID NO: 1 - DNA, 23 bp, Artificial Sequence
  The sequence is an immunostimulatory oligodeoxy nucleotide (ISS-ODN) No. 54076, endotoxin free, obtained from the Weizmann Institute, Israel
tccataacgttgcaaacgttctg

SEQ ID NO: 2 - DNA, 23 bp, Artificial Sequence
  The sequence is an immunostimulatory oligodeoxy nucleotide (ISS-ODN) No. 51997, endotoxin free, obtained from the Weizmann Institute, Israel
tccatgacgttcctgacgttctg

SEQ ID NO: 3 - protein, 24 aa, Artificial Sequence
  The sequence  is the exemplary peptide as described in Example 1, which was encapsulated using the inventive method.
VLGGGVALLRVIPALDSLTPANED

(19) United States
(12) Patent Application Publication
(10) Pub. No.: US 2006/0029655 A1
(43) Pub. Date: Feb. 9, 2006

(54) Method for preparation of vesicles loaded with biological material and different uses thereof

(75) Inventors: Yechezkel Barenholz, Jerusalem (IL); Eliezer Kedar, Jersusalem (IL); Aviva Joseph, Jerusalem (IL)

Correspondence Address: NATH & ASSOCIATES, 112 South West Street, Alexandria, VA, 22314, US

(73) Assignee: Yissum Research Development Company Of The Hebrew University Of Jerusalem, Jerusalem (IL)

(21) Appl. No.: 10/482,112

(22) PCT Filed: Jun. 25, 2002
(86) PCT No.: PCT/IL02/00506

§ 371 Date: Jul. 29, 2004

Related U.S. Application Data
(60) Provisional application No. 60/300,065, filed on Jun. 25, 2001.

(51) Int. Cl. A61K 9/127

(52) U.S. Cl. 424/450

(57) ABSTRACT

The present invention discloses a method for an efficient entrapment of active biological material in liposomes. The method is based on the steps of drying a suspension of liposome-forming lipids and then hydrating the dry composition obtained with an aqueous solution containing a biologically active material to be entrapped in high yield in the liposomes thus formed. The invention also concerns liposomal formulations produced by the method of the invention and their uses.

### FIELD OF THE INVENTION

This invention generally relates to liposomal formulations and in particular to a method for the preparation of liposomes loaded with biological material and to the different uses of the method and its products.

### PRIOR ART

The following is a list of prior art which is considered to be pertinent for describing the state of the art in the field of the invention.
(1) Lichtenberg D., and Barenholz Y in Methods of Biochemical Analysis (Glick D., is Ed.) Wiley NY pp.337462, 1988;
(2) Barenholz Y, and Crommelin D. J. A., in Encyclopeida of Pharmaceutical Technology (Swabrick J and Boylan J. C. Eds.) Vol. 9, Marcel Dekker NY pp. 1-39 (1994);
(3) U.S. Pat. No. 6,156,337;
(4) U.S. Pat. No. 6,066,331;
(5) C. Kirby and G. Gregoriadis [Bio/Technology, November 1984, pages 979-984;
(6) Van Uden J., and Raz, E. (ed.) in Springer Semis Immunopathol. 22:1-9 (2000);
(7) McCluskie, M. J., et al. Vaccine, 19:2657-2660 (2001);
(8) Horner, A. A., et al. Immunol Rev. 179,102-118 (2001);
(9) Klinman, D. M., et al. Springer Semin. Immunopathol. 22:173-183 (2000);
(10) Wagner, H., et al. Springer Semin. Immunopathol. 22:167-171 (2000);
(11) Alving, C. R. (1997) in New generation vaccines, 2^{nd} ed. (Levine, M. M., Woodrow, G. C., Kaper, J. B., and Cobon, G. S., eds.), Marcel Dekker, New York, pp.207-213;
(12) Kedar, E. and Barenholz, Y (1998) in The biotherapy of cancers: from immunotherapy to gene therapy (Chouaib S, ed.), INSERM Paris, pp. 333-362.

### BACKGROUND OF THE INVENTION

Several attempts have been made to use lipid vesicles formed by natural or synthetic phospholipids as vehicles for the administration of effective substances. Proposed clinical uses have included vaccine adjuvanticity, gene transfer and diagnostic imaging, but the major effort has been in the development of liposomes as non-targetable and targetable drug carriers in the treatment of malignancy, and infectious diseases such as fungal infections.

Amphotericin B, an effective but toxic antifungal, was the first liposomally formulated agent to be licensed for parenteral use in Europe.

Antitumor agents like adriamycin (doxorubicin) have also been incorporated into liposomes. DOXIL (liposomal doxorubicin) is the first liposomal drug approved for parenteral clinical use in the USA. Other liposomal formulations were developed as carriers for vaccines, adjuvants and biological response modifiers like cytokines and others.

Liposomes are also utilized as vehicles in the field of gene transfer [Kastel P. L., and Greenstein R. J., Biotechnol. Annu. Rev. 5:197-220 (2000)]. In another application, liposomes were used for the delivery of therapeutic proteins. N. Sakuragawa et al. [Thrombosis Research 38:681-685, (1985); Clinical Hematology 29(5):655-661 (1988)] report that liposomes containing factor VIII have been prepared for oral administration to patients suffering from von Willebrand's disease.

The encapsulation of factor VIII was carried out by dissolving the protein factor VIII concentrates in an aprotinin containing solution and transferred into lecithin coated flasks. After drying the flasks by rotation for 30 min under negative pressure liposomes were formed which entrapped factor VIII concentrates. The liposome dispersion was centrifuged yielding 40% of factor VIII entrapped in liposomes.

Another method for entrapment of drugs in liposomes is based on a procedure referred to by the term “dehydration-re-hydration”. This is described by C. Kirby and G. Gregoriadis [Bio/Technology, November 1984, pages 979-984]. In this preparation the entrapment was increased by using additional lipid and the use of cholesterol is described as having positive influence on drug entrapment.

Yet another method for loading vesicles with biological substances is described by 3.2.2 in U.S. Pat. Nos. 6,066,331 and 6,156,337. According to the method(s) described therein, liposomes loaded with biological structures, biopolymers and/or oligomers, are obtained by codying a fraction of an amphipathic material (liposome-forming lipids) in an organic solvent and a fraction of the biological structure(s), biopolymers and/or oligomers, from an aqueous medium.

The present invention aims for the providence of a novel method for efficient encapsulation (>60%) of biological material, particularly those being therapeutically active, into lipid membrane vesicles (liposomes).

A group of biological materials of interest according to the present invention are oligonucleotides and, especially, immunostimulatory oligodeoxy-nucleotides and their analogs (ISS-ODN or CpG motifs). Typically, ISS-ODN are short synthetic oligodeoxynucleotides (6-30 bases) usually containing an active 6-mer sequence that has the general structure of two 5′ purines, an unmethylated CpG dinucleotide, and two 3′ pyrimidines (Pu-Pu-CpG-Pyr-Pyr).

Bacterial DNA and its synthetic ISS-ODN are known to be potent stimulators of both innate immunity and specific adaptive immune responses, including direct activation of monocytes/macrophages, dendritic cells, NK cells and B cells. Further, bacterial DNA and its synthetic ISS-ODN induce the production of pro-inflammatory cytokines (e.g., IL-6, IL-12, IFNs, TNFα) and up-regulate the expression of MHC I, MHC II and co-stimulatory molecules [Van Uden J., and Raz, E. in Springer Semin. Immunopathol. 22:1-9 (2000)].

In animal studies, ISS-ODNs exhibit strong Thl and mucosal adjuvanticity to a wide range of antigens [McCluskie, M. J., et al. Vaccine, 19:2657-2660 (2001)] or allergens [Horner, A. A., et al. Immunol Rev. 179:102-118 (2001)]. Furthermore, pretreatment with ISS-ODN, even without concomitant administration of the relevant antigen, was shown to afford protection (for about 2 weeks) against subsequent infection with intracellular pathogens [Klinman, D. M., Springer Semin Immunopathol. 22:173-183 (2000)], indicating activation of innate immunity.

The immunostimulatory activity of ISS-ODNs requires cellular uptake by endocystosis following their binding to a cell receptor belonging to the Toll-like receptor family, TLR9. Endosomal acidification and digestion of the ODN followed by interaction with specific protein kinases results in rapid generation of reactive oxygen intermediates, leading to activation of MAPK and NF-κB pathways and subsequent is cytokine production (Chu, W., et al. Cell 103:909-918 (2000)].

In mice, doses of 50-100 μg/dose/mouse of soluble ISS-ODN, and in many cases two or more administrations are required to achieve the desired immunomodulatory effects. This relatively high dose and repeated administration, in theory, may cause adverse reactions resulting from the “cytokine storm” induced [Wagner, H., et al. Springer Semin. Immunopathol. 22:167-171 (2000)].

Liposomes can effectively entrap various drugs and biologicals, which are slowly released over an extended period of time in vivo, and are rapidly and efficiently taken up by macrophages and dendritic cells, suggesting that liposomes can serve as an efficient delivery system for biological material such as ISS-ODN-based vaccines [Alving, C R. (1997) in New generation vaccines, 2^{nd} ed. (Levine, M. M., Woodrow, G. C., Kaper, J. B., and Cobon, G. S., eds.), Marcel Dekker, New York, pp. 207-213; and Kedar, E. and Barenholz, Y. (1998) in The biotherapy of cancers: from immunotherapy to gene therapy (Chouaib S, ed.), INSERM, Paris, pp. 333-362].

Other groups of biological materials of interest according to the present invention are antigens (i.e., vaccines) and immunostimulatory cytokines (e.g., interleukin-2 [IL-2], granulocyte-macrophage colony-stimulating factor [GM-CSF], interferon γ [IFN-γ] It has been shown in several studies that liposomal delivery of vaccines and cytokines markedly enhance their bioactivity in animal models [Alving C. R. (1997) in New generation vaccines, 2^{nd} ed. (Levine, M. M., Woodrow, G. C., Kaper, J. B., and Cobon, G. S., eds.), Marcel Dekker, New York, pp. 207-213; Kedar, E. and Barenholz, Y. (1998) in The biotherapy of cancers: from immunotherapy to gene therapy (Chouaib S, ed.), INSERM, Paris, pp. 333-362; Gregoriadis, G., McCormack, B., Obrenovic, M., Saffie, R., Zadi, B. and Perrie, Y. Methods 19: 156-162 (1999)].

It should be noted, however, that in these studies, encapsulation in liposomes was carried out by various techniques which are time-consuming, and often result in a low encapsulation efficiency and low stability.

### SUMMARY OF THE INVENTION

The present invention is based on the surprising finding that step wise hydration of lipids, a priori freeze dried, with a solution containing biological material to be loaded into liposomes, results in a very effective loading (≧60%) of the material as compared to hitherto known loading methods.

Thus, according to a first of its aspects, the present invention provides a method for loading biological material in liposomes, the method comprises:
i) solubilizing (dissolving) at least one liposome-forming lipid in a solvent and drying the same to effect a dry liposome-forming lipid or a mixture of such lipids;
ii) providing an aqueous solution of biological material or of a mixture of biological material;
iii) hydrating the dry liposome-forming lipid(s) with the solution of biological material to yield liposomes loaded with said biological material.

The term “liposome” as used herein includes all spheres or vesicles of amphipathic substance that may spontaneously or non-spontaneously vesiculate, for example, phospholipids which are glycerides where at least one acyl group is replaced by a complex phosphoric acid ester.

The term “loading” means any kind of interaction of the biological substances to be loaded, for example, an interaction such as encapsulation, adhesion, adsorption, entrapment (either to the inner or outer wall of the vesicle or in the intraliposomal aqueous phase), or embedment in the liposome's membrane, with or without extrusion of the liposome containing the biological substances.

Also as used herein, the term “liposome-forming lipid” denotes any physiologically acceptable amphipathic substance that contains groups with characteristically different properties, e.g. both hydrophilic and hydrophobic properties or a mixture of such molecules, and which upon dispersion thereof in an aqueous medium form liposomal vesicles. As will be further elaborated hereinafter, this term refers to a single amphipathic substance or to a mixture of such substances. The amphipathic substance includes, inter alia, phospholipids, sphingolipids, glycolipids, such as cerebrosides and gangliosides, PEGylated lipids, and sterols, such as cholesterol and others.

The terms “dry” or “drying” refer to any manner of drying the liposome-forming lipids which results in the formation of a dry lipid cake. According to one preferred embodiment, drying is achieved by freeze drying, also referred to as lyophilizing. Alternatively, drying may be achieved by spray drying.

The term “biological material” used herein refers to any compound or polymer (e.g. biopolymer) or other biological structure having a biological effect on cells or cell constituent (e.g. enzyme, receptor). The biological material may be natural or synthetic and include, inter alia, active or inactive virions, bacteria or other pathogens, and biological cell structures (e.g., subcellular organelles such as ribosomes, membrane fractions, or mitochondriae, cell products (e.g., cytokines), and natural or synthetic biopolymers and/or natural or synthetic biooligomers (i.e., peptides, carbohydrates, and nucleic acids including DNA, RNA and oligonucleotides).

The term “solubilizing” which is used herein interchangeably with the term “dissolving” or “dispersing” may be achieved by a single use of the bulk aqueous medium with which said solubilization is achieved. However, this term preferably refers to step-wise addition of two or more aliquots of the said medium.

The method of the invention will at times be referred to in the following description by the term “post-encapsulation”, according to which dry lipids are hydrated with an aqueous solution containing the biological material. This is as opposed to the co-encapsulation technique. “Co-encapsulation” is an encapsulation method which includes codying the liposome-forming lipids and the biological material (co-lyophilized) after which they are co-hydrated with an aqueous medium. The co-encapsulation technique is described, inter alia, in U.S. Pat. Nos. 6,156,337 and 6,066,331.

One unique feature of the post encapsulation methodology disclosed herein is that it does not necessitate the freeze-dying of the biological material. As may be appreciated, there are numerous biological substances, e.g. proteins that serve as vaccine antigens, or enzymes, which are sensitive to lyophilization, leading to the deactivation of the biological substance. One example of such a sensitive vaccine is the influenza vaccine. In addition, according to the method of the present invention, the biological material does not need to be exposed to an organic solvent or detergent that may be destructive to its activity. For example, dissolution of the influenza virus hemagglutinin molecule in the presence of an organic solvent results in the dissociation of this trimeric protein into its monomers and consequently in loss of its biological activity (immunogenicity).

As indicated above and will be further shown in the following Examples, the method of the present invention enables to obtain vesicles with substantially high loading rate of the biological material (at least and preferably more than 60%). This feature is advantageous since it improves efficiency of treatment or prophylaxis with the biological material loaded into the liposomes as well as it enables to reduce the dose and frequency/number of composition administrations required in order to achieve a desired therapeutic effect.

Another feature of the method of the present invention is that since the lipid(s) substance(s) and the biological material are kept separately, it enables combinatorial formulations, i.e. the physician may prescribe and the pharmacist may formulate any combination of liposome-forming substance and biological agent, and upon need, the pharmacist can easily prepare the selected combination and prepare the desired formulation, according to the said simple and flexible method steps of the present invention.

Yet another feature of the present invention is that the freeze-dried lipids have a long shelf-life at 4° C. or room temperature, preserving their entrapment capability for over a year (as also exemplified in the following Example 4), and that the hydration of the lipids with the solution containing the biological material to form the liposomes is very simple and requires only several minutes. Therefore, the liposomal formulation can be readily prepared before treatment, ensuring high pharmaceutical stability of the formulation and without leakage of the entrapped material from the liposomes.

According to a second aspect, there is provided a combination of two compositions, including a first composition comprising dry liposome-forming lipids and a second composition comprising biological material, the combination intended for use in the preparation of a pharmaceutical composition comprising liposomal biological material.

The combination of the invention may be provided in the form of a package. Accordingly, the present invention also provides a package for the preparation of a pharmaceutical composition comprising:
(a) at least one composition of dry liposome-forming lipid(s);
(b) at least one composition of biological material;
(c) instructions for selection and use of (a) and (b) for the preparation of said pharmaceutical composition, said instructions comprising hydrating said dry liposome-forming lipid with an aqueous solution of said biological material, to yield a pharmaceutical composition comprising liposomes loaded with said biological material, and
(d) instructions prescribing adminison of said pharmaceutical composition to a healthy subject or to a patient in need of said composition.

According to another aspect of the invention, there is provided a pharmaceutical composition comprising as active ingredient a therapeutically lo effective amount of biological material loaded onto liposomes; the loaded liposomes being prepared by the method of the invention.

The pharmaceutically “effective amount”, including also a prophylactically effective amount, for purposes herein is determined by such considerations as are known in the art. The amount of the biological material must be effective to achieve a is desired therapeutic effect.

According to yet a further aspect of the invention there is provided a method for the prevention or treatment of a disease by administration to a subject in need an effective amount of the liposomes loaded with biological material according to the present invention.

The terms “prevention or treatment” or “treatment” as used herein refer to administering of a therapeutic amount of the liposome-loaded biological material which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, to prevent the disease form occurring, or a combination of two or more of the above. In addition, the term “treatment” in the context used herein refers to prevention of a disease from occurring. The treatment (also preventative treatment) regimen and the specific formulation to be administered will depend on the type of disease to be treated and may be determined by various considerations known to those skilled in the art of medicine, e.g. the physicians.

### DETAILED DESCRIPTION OF THE INVENTION

Liposomes can be classified according to various parameters. For example, when size and number of lamellae (structural parameters) are used, four major types of liposomes are identified: Multilamellar vesicles (MLV), small unilamellar vesicles (SUV), large unilamellar vesicles (LUV) and oligolamellar vesicles.

MLV form spontaneously upon hydration of dried phospholipids above their gel to liquid crystalline phase transition temperature (Tm). Their size and shape are heterogeneous and their exact structure is determined by their method of preparation [Barenholz, Y and Crommelin, D. J. A., (1994) ibid.]. In general, MLV have an aqueous and lipid components separated by bilayers.

SUV are formed from MLV by ultrasonic irradiation, high pressure homogenization, or by extrusion and are single bilayered (<100 nm). They are the smallest species with a high curvature and high surface-to-volume ratio and hence have the lowest capture volume of aqueous space to weight of lipid.

The third type of liposome according to this classification includes large unilamellar vesicles (LUV, ≧100 nm) having a large aqueous compartment and a single (unilamellar) lipid layer, while the fourth type of liposome includes oligolamellar vesicles (OLV), which are vesicles containing few lamellae (lipid bilayers). The LUV are formed mainly by extrusion.

Liposomes are formed from amphipathic compounds, which may spontaneously or non-spontaneously vesiculate. Such amphipathic compounds typically include triacylglycerols or trialkylglycerols where at least one acyl or one alkyl group is replaced by a polar and/or charged moiety, e.g. phospholipids formed by a complex phosphoric acid esters. Any commonly known liposome-forming lipids are suitable for use by the method of the present invention. The source of the lipid or its method of synthesis is not critical: any naturally occurring lipid, with and without modification, or a synthetic hosphatide can be used.

The lipidic substance may be any substance that forms liposomes upon dispersion thereof in an aqueous medium. Preferred liposome-forming amphipathic substances are natural, semi-synthetic or fully synthetic, molecules; negatively or positively charged lipids, phospholipids or sphingolipids, optionally combined with a sterol, such as cholesterol; and/or with lipopolymers, such as PEGylated lipids.

The liposome-forming lipids may include saturated or unsaturated amphiphiles. Non-limiting examples of such amphiphiles are phospholipids including, without being limited thereto, fully hydrogenated, partially hydrogenated or non-hydrogenated soybean derived phospholipids, egg yolk phospholipids, dimyristoyl phosphatidylcholine (DMPC), dimyristoyl phosphatidylglycerol (DMPG), other phosphatidylglycerols, phosphatidylinositols,_phosphatidylserines, sphingomeylins, and mixtures of the above. Another group of liposome-forming lipids are the cationic lipids, including, monocationic lipid, such as 1,2-dimyristoyl-3-trimethylammonium propane (DMTAP), 1,2-dioleoyl-3-trimethylammonium propane (DOTAP) and 1,2-distearoyl-3-trimethylammonium propane (DSTAP) and polycationic lipids, such as the speramine-based lipid N-[2-[[2,5-bis[(3-aminopropyl)amino]-1-oxopentyl]amino]ethyl]-N,N-dimethyl-2,3-bis[(1-oxo-9-octadecenyl)oxy]-1-propanaminium (DOSPA), which may be used either alone or in combination with cholesterol or with neutral phospholipids.

Examples of specific phosphatides are L-α-(distearoyl)phosphatidylcholine (lecithin), L-α-(diapalmitoyl)lecithin, L-α-phosphatidic acid, L-α-(dilauroyl)-phosphatidic acid, L-α(dimyristoyl)phosphatidic acid, L-α(dioleoyl)phosphatidic acid, DL-α(dipalmitoyl)phosphatidic acid, L-α(distearoyl)phosphatidic acid, and the various types of L-α-phosphatidylcholines and other phospholipids prepared from brain, liver, egg yolk milk, heart, soybean and the like, or synthetically, and salts thereof. Other suitable modifications include the controlled peroxidation of the fatty acyl residue cross-linkers in the phosphatidylcholines (PC), and in the other phospholipids, and the zwitterionic amphiphates, which form micelles by themselves or when mixed with the PCs such as alkyl analogues of PC.

According to one embodiment, lecithines (also known as phosphatidylcholines (PC)) are used, which are mixtures of the diglycerides of stearic, paimitic, and oleic acids linked to the choline ester of phosphoric acid. The lecithines are found in all animals and plants such as eggs, soybeans, and animal tissues (brain, heart, and the like) and can also be produced synthetically.

A preferred phospholipid combination according to the invention includes a mixture of DMPC and DMPG at a molar ratio of DMPC:DMPG between about 1:20 and 20:1. Such mixtures may be combined with cholesterol, and/or PEGylated lipids. PEGylated lipids are commercially available. Preferred PEGylated lipids include, without being limited thereto, negatively charged DSPE-PEG²⁰⁰⁰ [Haran, G., et al. Biochim. Biophys. Acta 1151:201-215 (1993)] or dihexadecyl phosphatidyl PEG²⁰⁰⁰ (DHP-PEG²⁰⁰⁰) [Tirosh, O., et al. Biophys. J. 74:1371-1379 (1998); U.S. Pat. No. 6,165,501], neutral PEG diacylglycerol, and PEG ceramides (Avanti Catalog). Another preferred lipid combination consists of DOTAP and cholesterol in a mole ratio of 1:2 to 20:1.

The lipids can vary in purity and can also be hydrogenated either fully or partially. Hydrogenation (partial or complete) reduces the level of unwanted peroxidation, and modifies and controls the gel to liquid/crystalline phase main transition temperature (Tₘ) which effects packing and leakage.

The liposomes may contain other lipid components, or a combination of lipid components. Such lipids include, but are not limited to, sterols (i.e., cholesterols), lipopolymers (i.e., PEGylated lipids), glycosphingolipids (i.e., gangliosides), and phosphatidyl ethanolamines.

The liposomes can be “tailored” to the requirements of any specific reservoir including various biological fluids, which maintain their stability without aggregation or chromatographic separation, and thereby remain well dispersed and suspended in the injected fluid. The fluidity in situ changes due to the composition, temperature, salinity, bivalent ions and presence of proteins. The liposomes can be used with or without any other solvent or surfactant.

A variety of methods for producing the different types of liposomes are known and available. Such methods include, inter alia:
1. hydrating a thin dried film of a phospholipid with an aqueous medium followed by mechanical shaking, ultrasonic irradiation and/or extrusion of the liposomes thus formed through a filter with a suitable pore size;
2. dissolving a phospholipid in a suitable organic solvent, miring with an aqueous medium followed by removal of the solvent;
3. use of a gas above its critical point (i.e., freon and other gases such as CO₂ or mixtures of CO₂ and other gaseous hydrocarbons) or
4. preparing of lipid-detergent mixed micelles followed by lowering the concentration of the detergent to a level below its critical concentration at which liposomes are formed [Lichtenberg D and Barenholz Y (1988) ibid.].
5. hydrating dry liposomes, loaded with an active agent, with an aqueous medium, referred to as the CO loading method (U.S. Pat. No. 6,066,331, U.S. Pat. No. 6,156,337).

One obstacle when using liposomes as a drug delivery tool, are the potential destructive/inactivating effect of the loading process on the biological material to be loaded into the liposome, and the efficiency of loading of the biologically effective material. For water-soluble expensive drugs passively loaded into the intraliposomal aqueous phase, the hitherto best loading is ≦60%. Non-efficient loading leaves a large amount of the drug un-encapsulated, and when the drugs are toxic and/or expensive this un-encapsulated drug is a major drawback. Therefore, an additional step of removal of the free drug is required, which adds unwanted handling and cost to the process of preparation of liposome formulation.

The present invention provides a novel and simple method for preparing liposomes efficiently loaded (i.e. at least 60% loading) with the biological material. The method of the invention comprises:
i) solubilizing at least one liposome-forming lipid in a solvent and drying same to effect a dry lipid or a dry mixture of lipids;
ii) providing a solution of biological material or of a mixture of biological materials; and
iii) hydrating the dry lipid(s) with said solution of biological material to yield liposomes loaded with biological material.

As will be shown in the following specific Examples, the method of the invention provides a highly effective entrapment of the biologically active material in the liposomes, typically greater than 60% (from the initial amount of biological material employed for loading).

According to the present invention, the liposome-forming lipids are preferably freeze dried, i.e. by lyophilization thereof, resulting in a powder with a unique arrangement of the lipids enabling the effective loading into the liposomes of the biological material upon hydration.

The solvent according to the invention is any solvent with which the amphipathic substance (lipid) may be solublized, and includes polar solvents such as tertiary butanol or apolar solvents, such as cyclohexane.

The active material entrapped by the liposomes according to the method of the invention is a biological material or a mixture of biological materials including, inter alia, biological cell structures or cell products, natural or synthetic biopolymers and/or oligomers (e.g. amino acids or nucleic acid sequences).

The biological cell structures are preferably cell membranes, ribosomes, or mitochondriae, while the cell products, biopolymers and oligomers, are preferably enzymes, proenzymes, hormones, and cofactors; also live or inactivated viruses or virus surface antigens, antigens, antibodies, complement factors, live or inactivated bacteria, bacterial fragments, bacterial surface antigens, other pathogens and their products, cytokines, growth factors, natural or synthetic nucleotides, DNA, mRNA, rRNA, tRNA, antisense DNA, antisense RNA, or inhibitory RNA (iRNA).

According to one embodiment, the biological material is an oligodeoxynucleotide (ODN), preferably, an immunostimulatory oligodeoxynucleotide sequence (ISS-ODN). As explained herein, such sequences are known to enhance the immune response (act as immunoadjuvants) and, therefore, are of a therapeutic value.

One preferred ODN according to the invention is the endotoxin-free phosphorothioate ISS-ODN. According to yet another embodiment, the ODN is the anti-sense anti-Bcl2 known to inhibit expression of the Bcl2 protein, thereby enhancing cell apoptosis [Meidan V. M., et al. Biochimica et Biophysica Acta 1464:251-261 (2000)].

According to the method of the invention, it is advisable to keep the biopolymers and oligomers in a medium having an ionic strength corresponding to up to 5% sodium chloride, with or without cryprotectant, which is a pharmaceutically acceptable agent, such as lactose, sucrose or trehalose. Thus, the aqueous solution according to the present invention is a physiologically acceptable aqueous medium employed by the method of the invention for solubilizing, dissolving or dispersing the biological material, typically selected from the group consisting of 0.9% NaCl by weight (Saline), buffered Saline such as phosphate-buffered Saline (PBS), 5% dextrose, buffered dextrose, 10% sucrose and buffered sucrose, and any combination of the same. Alternatively, the biological material is solubilized in pyrogen-free sterile water (at times referred to as ‘water for injection’) and after hydration of the dry lipids, the resulting dispersion is adapted to the physiological conditions suitable for administration.

According to a second aspect, there is provided a combination of two compositions, including a first composition comprising dry liposome-forming lipids and a second composition comprising biological material, the combination intended for use in the preparation of a pharmaceutical composition comprising liposomes loaded with biological material.

The combination of the invention may be provided in the form of a package. Accordingly, the present invention also provides a package for the preparation of a pharmaceutical composition comprising the combination of the at least one first composition comprising dry liposome-forming lipids; and of at least one second composition comprising biological material (either dry or in an aqueous solution); and instructions for use of the first and second compositions for the preparation of said pharmaceutical composition, said instructions comprise hydrating the dry lipid(s) with said aqueous solution comprising the biological material, to obtain liposomes loaded with the biological material; and further instructions prescribing administration of the pharmaceutical composition to a subject in need.

Within the package of the invention the dry lipids and the biological material are each contained in a separate vial. The kit may thus contain more than one type of composition of dry lipid in separate vials and more than one biological material, the instructions for selection and use of the different compositions (i.e. the first and second composition) will depend on the specific liposome/biological material formulation of interest. These instructions may be addressed to the physician, to the pharmacist or even to the individual in need.

The package may further comprise an aqueous medium, e.g. a physiologically acceptable aqueous medium, with which the biological material can be dissolved or diluted prior to use. Alternatively, the aqueous medium may be obtained separately, as it is typically a commercially available medium. Selection of the medium suitable for use will depend on considerations known to those versed in the art and, therefore, do not need to be further discussed herein.

According to one embodiment, the package comprises two or more compositions of said first composition comprising dry liposome-forming lipid(s) and two or more of said second compositions of biological material, thereby enabling to construct different combinations of formulations according to instructions prescribed by the medical practitioner. The package may be for use by the physician, by the pharmacist or, at times, by the subject in need of the liposomal formulation.

According to a further aspect of the invention, there is provided a pharmaceutical composition comprising as active ingredient a therapeutically effective amount of liposomes loaded with a biological material and optionally a pharmaceutically acceptable additive, the loaded liposomes being prepared by the method of the invention.

In fact, the pharmaceutical composition of the invention is basically the liposomal formulation obtainable by the method of the invention but adapted for administration to the individual in need of a treatment or prevention of specified is disease.

The active ingredient of the present invention (i.e. the liposomes loaded with biological material) is administered and dosed in accordance with good medical practice, taking into account the nature of the biological material, the clinical condition of the treated individual, the site, route and method of administration, scheduling of administration, individual's age, sex, body weight and other factors known to medical practitioners.

The pharmaceutical composition of the invention may be administered in various ways. It may be formulated in combination with physiologically acceptable diluents, excipients, additives and adjuvants, as known in the art, e.g. for the purposes of adding flavors, colors, lubrication or the like to the liposomal formulation.

The pharmaceutically acceptable diluent/s, excipient/s, additive/s employed according to the invention generally refer to inert, non-toxic substances which preferably do not react with the liposomal formulation of the present invention.

Yet, the composition of the invention may comprise a combination of biological active agents. The additional biological agents may be in a free form or also encapsulated in liposomes (together or separated from the liposomes containing the other biological material/biological or pharmacological active material).

When the biological material is, for example, an ISS-ODN (an immuno-adjuvant), it is preferably administered in combination with one or more antigens. The antigens may be co-encapsulated with the ISS-ODN in the same liposomes, encapsulated in separate liposomes, or be in a free form (e.g. soluble or part of an emulsion). When the ISS-ODN and the antigen/s are separate, they may be administered simultaneously, or concomitantly within a predefined time interval. The antigen may be, inter alia, derived from a killed or modified (e.g. genetically) organism or virus.

The pharmaceutical composition can be administered orally, intranasally, or parenterally, including intravenously, intraarterially, intramuscularly, intra-peritoneally, intradermally, subcutaneously, intrathecally, and by topical delivery and infusion techniques. Yet further, the pharmaceutical composition of the invention may be made into aerosol formulations for administration by inhalation. Such aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. The manner of administration will depend on different considerations known to the man of the art (e.g. on the type of vaccine to be loaded into the liposome).

Finally, the present invention concerns a method for the prevention or treatment of a disease, the method includes administration to a subject in need an effective amount of the liposome-loaded biological material of the invention.

According to a preferred embodiment, the dosage for said treatment will include up to 2,000 mg of loaded vesicles measured by lipid per kg body weight of the treatment subject. It should be noted, however, that the accurate dosage can vary dramatically, the variation depends on e.g. the type and efficacy of the biological material entrapped by the liposome, the efficiency of encapsulation (albeit being high with the method of the invention), the route of administration and the like. The respective parameters may be easily optimized by those skilled in the art and can thus be regarded as being routine experiments.

The invention will now be further explained by the following non-limiting examples. While the foregoing description describes in detail only a few specific embodiments of the invention, it will be understood by those skilled in the art that the invention is not limited thereto and that other variations in form and details may be possible without departing from the scope and spirit of the invention as defined by the claims, which are to be read as included within the disclosure of the specification.

### SPECIFIC EXAMPLES

### Example 1

### Peptide-Loaded Liposomes

The following is an example of encapsulation of a peptide having the amino acid sequence: Val-Leu-Gly-Gly-Gly-Val-Ala-Leu-Leu-Arg-Val-Ile-Pro-Ala-Leu-Asp-Ser-Leu-Thr-Pro-Ala-Asn-Glu-Asp. The lipids employed for the different types of liposomes formed were DMPC, DMPG and cholesterol. Three types of liposome preparations were formed, for the purpose of comparison of the method of preparation of the present invention with other hitherto known methods. The three encapsulation methods employed are designated herein as post encapsulation (the method of the present invention); co-encapsulation and dehydration-rehydration. (the liposomes formed by the latter method are also referred to as the dehydration-rehydration vesicles (DRV)).

Liposomal Preparations

1. Post encapsulation: A lyophilized mixture of lipids (lipid:peptide w/w ratio varies as indicated in the following composition description) was hydrated with the peptide, a priori dissolved in an aqueous medium, such as distilled water, 0.9% NaCl (Saline) and/or 5% dextrose. In particular, the lipids were dissolved in tertiary butanol and freeze dried by lyopllization over night. The lipid cake formed was then rehydrated stepwise at room temperature with the peptide solution and vortexed vigorously for about 1 min.

2. Co-encapsulation: The solubilized lipids and peptide were co-lyophilized overnight and then hydrated with 0.9% Saline and/or 5% dextrose.

3. DRV: Lyoplilization of the peptide, a priori mixed with extruded (100 nm) liposomes in distilled water, to form a powder, followed by hydration of the powder with 0.9% Saline and/or 5% dextrose [Kirby C, and Gregoriadis G.Biotechnology 2: 979-84 (1984)).

In all preparations the lipid:peptide ratio (w/w) was optimized to 100:1.

Four lipid compositions were employed in the present example:
(i) DMPC alone;
(ii) DMPC:DMPG at a mole ratio of 9:1;
(iii) DMPC:Cholesterol at a mole ratio of 6:4; and
(iv) DNPC:DMPG:Cholesterol at a mole ratio of 9:1:6.5.

Twenty four types of peptide-loaded liposomal compositions were prepared depending on the method of encapsulation and the aqueous medium in which the lyophilized material was hydrated. As control, empty liposomes (i.e. without peptide) were prepared according to the post encapsulation procedure. Table 1 summarizes the different peptide-loaded liposomal compositions obtained and the encapsulation efficiency. Each liposomal composition was designated with a batch number: batches 1-12 hydration with an aqueous solution containing 0.9% Saline and batches 13-24 hydration with an aqueous solution containing 5% dextrose.

For the preparation of the different liposomal compositions, vials containing either co-lyophilized lipid and peptide, peptide or lipid alone were prepared. Each vial-powder contained 0.6 mg peptide. The peptide was filter-sterilized (0.2μ, Gelman Sciences, No. 4187) without loss. All compositions were prepared under sterile conditions.

Encapsulation Efficiency Measurement

Un-encapsulated (free) peptide was separated from the MLV-associated (or DRV-associated) peptide by centrifugation at 105,000 g for 30 min. at 4° C. using a TL 100 Beckman centrifuge. The supernatant was used for determination of the un-encapsulated peptide. To test stability of encapsulation, the liposome precipitate was washed with the same solution (as in the first time). The centrifugation was repeated and the level of the peptide in the wash was determined. The level of peptide encapsulation was determined by fluorescence assay, using a fluorescamine-labeled peptide [Bolikeun et al. Biochim. Biophys Acta 155:213-220 (1973)].

Results and Conclusions

The partition coefficient of the peptide between octanol and water two-phase system at different pHs (5, 7, and 8) was first determined. Accordingly, a solution of 0.1 mg/ml peptide was prepared with either sodium acetate buffer (5 ml, pH 5.0) or in 5 mM boric acid (1 ml, pH 7.0 or 8.0). The solution was mixed with octanol for 1 hr, after which aliquots of 100 μl and 200 μl were withdrawn from the aqueous phase (the lower phase) for determination of the partition coefficient. Almost 100% of the peptide partitioned into the aqueous phase, indicating low hydrophobicity of the peptide. This, together with the fact that the ratio of negatively- to positively-charged amino acid residues in the peptide is 3 to 1, suggests that the encapsulated peptide probably resides in the intaliposomal aqueous phase and not associated with the liposome membrane. The encapsulation efficiency and other features of the liposomes formed are summarized in the following Table 1.

TABLE 1
Encapsulation efficiency of a synthetic peptide, using different liposome
compositions and encapsulation methods

| Sample No. | | | | 1st wash, % | 2nd wash, % |
|---|---|---|---|---|---|
| and hydration | Phosphoilpid | Lipid | Preparation | Pep. in upper | Pep. in upper |
| solution | Cons. mM | composition | method | phase | phase |
| 0.9% NaCl | | | | | |
| 1 | 166.48 | I | Post | 67.28 | 14.08 |
| 2 | 150.44 | II | Post | 71.50 | 13.44 |
| 3 | 136.62 | III | Post | 78.67 | 3.92 |
| 4 | 131.30 | IV | Post | 14.50 | 0.77 |
| 5 | 185.66 | I | Co | 75.46 | 18.77 |
| 6 | 176.80 | II | Co | 58.65 | 28.03 |
| 7 | 140.40 | III | Co | 69.03 | 3.63 |
| 8 | 133.32 | IV | Co | 34.21 | 3.08 |
| 9 | 186.90 | I | DRV | 87.74 | 18.77 |
| 10 | 133.20 | II | DRV | 64.50 | 28.03 |
| 11 | 143.80 | III | DRV | 67.38 | 1.54 |
| 12 | 126.06 | IV | DRV | 37.85 | 1.87 |
| 5% dextrose | | | | | |
| 13 | 154.20 | I | Post | 47.10 | 45.59 |
| 14 | | II | Post | *hydrogel | *hydrogel |
| 15 | 152.60 | III | Post | 54.97 | 2.27 |
| 16 | 128.60 | IV | Post | 13.31 | 9.03 |
| 17 | 180.20 | I | Co | 64.59 | 36.10 |
| 18 | | II | Co | *hydrogel | *hydrogel |
| 19 | 141.20 | III | Co | 76.67 | 9.26 |
| 20 | 73.80 | IV | Co | 45.54 | 6.23 |
| 21 | 159.20 | I | DRV | 67.18 | 26.69 |
| 22 | | II | DRV | *hydrogel | *hydrogel |
| 23 | 130.60 | III | DRV | 72.28 | 1.36 |
| 24 | 100.60 | IV | DRV | 31.00 | 8.91 |

*hydrogel was formed

Table 1 shows that the best encapsulation (77%-85% encapsulation, samples no. 4 and 16) was obtained with a lipid composition of DMPC:DMPG:Chol, 9:1:6.5 (mole ratio) using the Post-encapsulation preparation method. Both cholesterol and DMPG were required in order to optimize encapsulation.

Further, in the presence of dextrose the liposome dispersions containing the peptide were more viscous than those prepared in 0.9% NaCl. Interestingly, for the 9:1 DMPC/DMPG liposomes in 5.0% dextrose the liposome dispersion formed a hydrogel.

### Example 2

### Liposomes Loaded with Immunostimulatory Oligonucleotides (ISS-ODNs) as Adjuvants for Influenza Vaccine

Materials and Reagents

Influenza subunit vaccine (HN)—A subunit preparation containing mainly the viral surface proteins hemagglutinin (H) and neuraminidase (N), 80-90% and 5-10% (w/w), respectively, derived from influenza A/New Caledonia/20/99 (H1N1) was provided by Dr's. IL Glück and R. Zurbriggen, Berna Biotech, Bern, Switzerland.

Dimyristoyl phosphatidylcholine (DMPC)—Lipoid PC 14:0/14:0 562157 (Lipoid GmbH, Ludwigshafen, Germany)

Dimyristoyl phosphatidylglycerol (DMPG)—Lipoid PG 14:0/14:0 602035-1 (Lipoid GmbH, Ludwigshafen, Germany)

ISS-ODN—Endotoxin-free (1<ng/mg DNA) phosphorothioate ISS-ODN No. 54076 (TCCATAACGTTGCAAACGTTCTG) and No. 51997 (TCCATGACGTTCCTGACGTTCTG), both dissolved in distilled water, were obtained from The Weizmann Institute, Rehovot, Israel.

Methods of Preparation

Preparation of Soluble HN

The subunit vaccine preparation was diluted in sterile phosphate-buffered saline (PBS pH 7.4) for injection (0.5 μg per dose).

Preparation of Liposomal ISS-ODN (Lip ISS-ODN)

ISS-ODNs were encapsulated in large (mean diameter 1400±200 nm) multilamellar vesicles (MLV) composed of DMPC and DMPG (DMPC:DMPG, 9:1 mole ratio), at a lipid:ODN ratio of 50:1-500:1 (w/w), under aseptic conditions as follows: The phospholipids were dissolved in tertiary butanol and freeze dried by lyophlization over night. The lipid powder (lipid cake) was then rehydrated at room temperature with the ODN solution. To ensure efficient encapsulation, ODN solution was added in a minimal volume (e.g. for 10 mg-30mg lipid, 25-50 μl of ODN solution was added). This was then vortexed vigorously for about 1 min. until a paste was obtained. The paste was then gradually diluted further by vortexing with sterile PBS or Saline to obtain the required concentration. This method corresponds to the post encapsulation method of the present invention.

To determine encapsulation efficiency, the liposomal preparation was centrifuged at 4° C., for 1 hr. at 45,000 rpm. The liposome precipitate and the supernatant (containing non-encapsulated ODN and traces of small liposomes) were subjected to a 2-phase lipid extraction procedure [Bligh, E. J. and Dyer, W. J. (1959) Canadian J. Biochem. Physiol. 37:911-917]., and the amounts of free and encapsulated ODN and liposomal phospholipids were assessed by organic phosphorus determination [[Barenholz, Y. and Amselem, S. (1993) in Liposome technology, 2^{nd} ed., Vol I. (Gregoriadis G, ed.), CRC Press, Boca Raton, Fla., pp. 501-525 (1993)]. The lipid integrity of freshly prepared Lip ISS-ODN was analyzed by thin layer chromatography (TLC) and was found to be high and identical to that of the lipid raw material (above 98%).

Using the following ratios (w/w) of lipid:ISS-ODN—50:1, 100:1, 300:1 and 500:1, the mean encapsulation efficiency (of 3 experiments) was 60, 75, 90 and 95%, respectively. No significant ODN leakage (<10%) from the liposomes was found after storage for three months at 4° C. To avoid overloading the mice with extra lipids, which can cause nonspecific immune stimulation [Kedar, E., et al. J. Immunother. 23:131-145 (2000)], the formulation prepared at a 100:1 (w/w) lipid:ODN ratio (mean encapsulation efficiency, 75%) was chosen for vaccination.

In a representative experiment, BALB/c mice (4/group) were vaccinated once, intramuscularly, with 0.5 μg free antigen (HN), alone and combined with free or liposomal ISS-ODN (No. 54076, or No. 51997), 10 μg each. The humoral response: hemagglutination-inhibiting (HI) antibodies and antigen-specific IgG1 and IgG2a were tested 4 weeks post-vaccination. HI test was carried out on individual sera, whereas Ig isotypes were tested by ELISA on pooled serum samples.

Results and Conclusions

As can be seen in the following Table 2, free antigen (group 2) induced very low HI and IgG2a titers, and both un-encapsulated ODNs markedly increased these titers (groups 3,5). Liposomal ISS-ODNs (groups 4, 6) were 2-7 times more potent than the corresponding free (non-liposomal) ODNs. In addition, whereas the response induced by free HN alone was a Th2-type (IgG2a/IgG1 ratio=0.04), the ODNs, free and liposomal, elicited a Th1-biassed response (IgG2a/IgG1 ratio ≧2). These data indicate that liposomal delivery of ISS-ODN potentiates the inherent immunoadjuvant-activity of ISS-ODN and preserve their Th1 adjuvanticity.

TABLE 2
Comparison of free and liposomal ISS-ODNs as adjuvants
for influenza vaccine: HI, IgG1 and IgG2a titers 4 weeks
post vaccination

| | Mean | Mean | IgG2a/IgG1 | Mean |
|---|---|---|---|---|
| Vaccine | IgG1 titer | IgG2a titer | ratio | HI titer^{a} |
| 1. None | <10 | <10 | — | 5 (0) |
| 2. HN alone | 1500 | 60 | 0.04 | 9 (0) |
| 3. HN + free ODN 1^{b} | 900 | 1500 | 1.7 | 52 (75) |
| 4. HN + lip ODN 1 | 2000 | 2800 | 1.4 | 140 (100) |
| 5. HN + free ODN 2^{c} | 45 | 700 | 15.5 | 31 (50) |
| 6. HN + lip ODN 2 | 1500 | 3500 | 2.3 | 210 (100) |

^{a}In parentheses, % seroconversion (% of mice with an HI titer ≧40).
^{b}ODN 54076;
^{c}ODN 51997.

### Example 3

### Liposomal Encapsulation of Antisense Bcl-2 (Lip Bcl-2)

The POST encapsulation method was applied for encapsulation of antisense to Bcl-2, the steps of which are the same as those described in connection with POST encapsulation of ISS-ODN. Encapsulation was performed at lipid:Bcl-2 ratios of 100:1 and 300:1 (w/w), yielding encapsulation efficacy of 78% and 74%, respectively. Encapsulation efficiency was determined as described herein before in connection with ISS-ODN.

### Example 4

### Liposomal Encapsulation of Influenza HN Antigens (Lip HN) in Various Liposomal Formulations

Materials and Reagents

Lipids

The lipids used for the preparation of the MLV liposomes included DMPC, DMPC/DMPG (9/1 mole ratio) as in Example 2. Additional formulations included DMPC/Cholesteral (Chol) (6/4 mole ratio), and the cationic liposomes consisting of: DMTAP (dimyristoyl-trimethylammonium propane)/Chol (1/1 mole ratio), DSTAP:(distearoyl-trimethylammonium propane)/Chol (1/1 mole ratio), DOTAP (dioleoyl-trimethylammonium propane)/Chol (1/1 mole ratio), DCCHOL (dimethylaminoethane-carbamol-cholesterol)/DOPE (dioleoyl-phosphatidylethanolamine) (1/1 mole ratio), and DDAB (dimethyldiocta decylammonium bromide)/Chol (1/1 mole ratio),

Influenza Antigens

Subunit (HN) antigen preparations derived from A/Beijing/262/95 (H1N1), A/Sydney/5/97 (H3N2), A/New Caledonia/20/99 (H1N1), A/Panama/2007/99 (H3N2), and B/Yamanashi/166/98 were obtained from Dr's R Zurbriggen and R. Glück, Berna Biotech, Bern, Switzerland. They were diluted in 0.9% NaCl prior to encapsulation.

Methods of Preparation

HN-loaded large multilamellar vesicle REV) (mean diameter, 1.5 μm) were prepared by using the POST-encapsulation method as described above in connection with preparation of Lip ISS-ODN, by adding HN subunits to the dry lipid cake.

In short, vials of 10-100 mg of various phospholipids' mixtures (see Tables 3, 5 for details), suspended in tertiary-butanol, were frozen and then lyophylized over night to form the dry lipid cake. Upon need, the dry lipid was hydrated with the subunit (HN) vaccine preparations (using 1, 2, or 3 strains, see materials and methods) by adding the soluble HN subunits at a lipid:HN ratio of 300:1 (w/w) in increments of 50 μl and vortexing vigorously. The liposomes were then suspended in sterile saline or PBS.

Encapsulation efficiency was assessed as follows: Liposomes were diluted with D₂O (1/1 v/v) and centrifuged at 30° C. for 45 min. at 14,000 rpm in an Eppendorf 5417 R centrifuge. Under these conditions, the liposomes float on top of the dense D₂O, while most of the unencapsulated antigen precipitates. The supernatant containing the liposomes and traces of free antigen was collected and spun at 4° C. for 60 min. at 14,000 rpm. Under these conditions the liposomes precipitate while most of the free antigen remains in the supernatant. The protein concentration of the antigen precipitate and of the latter supernatant (both containing the non-encapsulated antigen) and in the liposomal fraction (containing the entrapped antigen) was determined using a modified Lowry protein concentration determination assay [Peterson G. L., Methods Enzymol. 91:95-119 (1983)]. Recovery is >95% and precision is ˜90%.

Results and Conclusions

In the first experiment (Table 3), the subunit (HN) preparations were encapsulated in three formulations of neutral (DMPC, DMPC/Chol) or negatively-charged (DMPC/DMPG) liposomes using the POST technique (the present invention). As can be seen, 60-100% of the antigen was encapsulated, depending on viral strain and formulation. This high level of HN encapsulation was equal to, or better than, that obtained by the CO technique or by using DRV. However, whereas the immunogenicity of HN encapsulated by the POST technique was fully retained, it was markedly reduced (up to 90%, especially of influenza B strains) using the CO technique or DRV (data not shown). The lipid integrity (determined by TLC) of the HN-loaded liposomes was above 98%.

TABLE 3
Liposome encapsulation of influenza subunit vaccines in
various non-cationic liposomal formulations

| Formulation | % HN encapsulation^{a} |
|---|---|
| DMPC | 87-93 |
| DMPC/DMPG (9/1 mole ratio) | 80-100 |
| DMPC/Chol (6/4 mole ratio) | 60-90 |

^{a}Range of 3 experiments, using subunit vaccines derived from A/New Caledonia and B/Yamanashi strains.

The immunogenicity of free and liposomal (NMPC/DMPG, 9/1 mole ratio) divalent influenza subunit vaccine was tested in BALB/c mice following a single intraperitoneal administration (0.5 μg HN of each viral strain). The response (serum HI titer) was tested 30 days post-vaccination. As can be seen in Table 4, the liposomal antigen (Lip HN) was considerably more immunogenic than the free antigen for the two A strains.

TABLE 4
The anti-hemagglutinin response of BALB/c mice vaccinated
intraperitoneally with free/liposomal divalent influenza subunit vaccine

| | HI titer (mean ± SD) against: | |
|---|---|---|
| Vaccine | A/Sydney/5/97 | A/Beijing/262/95 |
| (n = 5/group) | (H3N2) | (H1N1) |
| HN | 32 ± 39 (40%)^{a} | 5 ± 8 (0%) |
| Lip-HN | 320 ± 160 (60%) | 24 ± 32 (40%) |

^{a}In parentheses, % seroconversion (% of mice with an HI titer of ≧40).

Vaccination Against Influenza by Intranasal Administration of Influenza Subunit Vaccine (HN) Entrapped in Various Formulations of Cationic Liposomes

In an additional experiment, female (n=5/group) Balb/c mice were vaccinated on days 0 and 7 (10 μL/nostdil/dose), using 3 μg of a subunit vaccine (HN) derived from influenza A/New Caledonia/20/99 (H1N1). The antigen was administered either in soluble form or entrapped (using the “POST” technique) in large (mean diameter˜1.5 μm) multilamellar liposomes (Lip) consisting of various cationic phospholipids, with and without cholesterol (1/1 mole ratio), as indicated in Table 5. The lipid/BN (protein) w/w ratio was 300/1 and encapsulation efficiency was ˜80%. Cholera toxin (CT), a standard mucosal adjuvant in animal studies, was used as a positive control. Mice were bled 28 days after vaccination and sera were tested for hemagglutination-inhibiting (HI) antibodies (tested on individual mice) and by ELISA for antigen-specific IgG1 and IgG2a antibodies (tested on pooled sera of each group), starting at 1/10 serum dilution.

As can be seen in Table 5, free antigen (group 2) was completely incapable of inducing any response. In contrast, encapsulated antigen was highly efficient in inducing HI, IgG1 and IgG2a Abs, particularly when encapsulated in liposomes comprising DOTAP:CHOL (group 5), followed by DMTAP:CHOL (group 3). The antibody response obtained by the former formulation was even considerably higher than that obtained with CT (group 8), known to be the most powerful, yet toxic (not allowed for human use), mucosal adjuvant.

The induction of such a strong systemic immune response following intranasal (mucosal) vaccination, without the need for an additional adjuvant, is of particular interest and indicates that certain cationic liposome formulations serve both as an efficient delivery system for the antigen and as a powerful mucosal adjuvant. The DOTAP/CHOL and DMTAP/CHOL formulations were also highly effective upon intramuscular vaccination (data not shown).

It should be noted that whereas HN antigen encapsulated in neutral liposomes (DMPC, DMPC/CHOL) or negatively-charged liposomes (DMPC/DMPG) (see Tables 3, 4) is more immunogenic than free antigen when administered parenterally (i.p., i.m.), such liposomal antigen formulations are much less effective when administered intranasally (data not shown), thus emphasizing the superiority of the cationic liposomes prepared by the “POST” method for intranasal (mucosal) vaccination.

TABLE 5
Induction of anti-influenza humoral response in mice by free or liposome
encapsulated subunit vaccine administered intranasally

| | | HI titer mean ± | | | |
|---|---|---|---|---|---|
| | | SD(% | ELISA titer | | |
| | Group^{a} | seroconversion)^{b} | IgG1 | IgG2a | IgG2a/IgG1 |
| 1. | Normal | 5 ± 0 (0%) | 0 | 0 | — |
| 2. | HN | 6 ± 2 (0%) | 0 | 0 | — |
| 3. | Lip (DMTAP:CHOL)-HN | 152 ± 96 (100%) | 200 | 20 | 0.10 |
| 4. | Lip (DSTAP:CHOL)-HN | 28 ± 29 (40%) | 10 | 0 | 0.00 |
| 5. | Lip (DOTAP:CHOL)-HN | 576 ± 128 (100%) | 7000 | 1000 | 0.14 |
| 6. | Lip (DCCHOL:DOPE)-HN | 18 ± 7 (0%) | 0 | 0 | — |
| 7. | Lip (DDAB:CHOL)-HN | 136 ± 32 (100%) | 100 | 0 | 0.00 |
| 8. | HN + CT 1 μg | 122 ± 83 (60%) | 500 | 10 | 0.02 |

^{a}CHOL = Cholesterol; DMTAP: = Dimyristoyl-Trimethylammonium-Propane; DSTAP = Distearoyl-Trimethylammonium-Propane; DOTAP = Dioleoyl-Trimethylammonium-Propane; DCCHOL:DOPE = Dimethylaminoethane-Carbamol-Cholesterol:Dioleoyl-Phosphatidylethanolamine at a mole ratio of 1:1; DDAB = Dimethyldioctadecylammonium Bromide.
^{b}Tested by hemagglutination inhibition. Values in parentheses represent % seroconversion (% of mice with an HI titer ≧ 40). 0 denotes <10.

Effect of Long-Term Storage of Freeze-Dried Lipids on Liposomal Encapsulation Efficacy of Influenza HN Antigens and on Chemical Integrity of the Lipids

HN-loaded large multilamellar vesicles were prepared by the POST encapsulation technique, using DMPC/DMPG (9/1 mole ratio) dissolved in tertiary butanol then freeze-dried overnight and stored for 20 months at 4° C. prior to hydration with the HN solution (derived from 3 influenza strains). Lipid hydrolysis was below 5%, and % HN encapsulation (60-80%, depending on strain) and mean size of the liposomes (1-1.5 μm) were identical to those of freshly freeze-dried lipids. This liposomal vaccine was as efficacious, in mice, as a lo vaccine prepared from freshly freeze-dried lipids. These findings indicate that large batches of freeze-dried lipids can be prepared and stored until use.

### Example 5

### Liposomal Encapsulation of Recombinant Human Interleukin 2 (Lip IL-2)

IL-2 is a potent immunostimulating cytokine and is being used in the is treatment of patients with metastatic melanoma, metastatic renal carcinoma, and AIDS. IL-2 (Chiron, USA, 18×10⁶ IU/mg) was encapsulated in DMPC/DMPG (9:1 mole ratio) MLV liposomes (mean diameter, 1.2-1.5 um), using the POST-encapsulation technique as disclosed herein, for example, in connection with the preparation of Lip ISS-ODN, at a lipid:IL-2 ratio of 125:1-300:1 (w/w). Encapsulation efficiency was 80-90% as determined by bioassay (Kedar E., et al. J. Immunother 23:131-145 (2000)]. The liposomal IL-2 was suspended in PBS and stored at 4 C for up to 6 months. IL-2 leakage at 3 months was less than 10% and at 6 months 20-30%.

Liposomal IL-2 proved to be a much more potent vaccine adjuvant than soluble IL-2 in mice upon co-administration with influenza vaccines. In a representative experiment shown in Table 6, free or liposomal trivalent influenza subunit (BM vaccine was administered once, intraperitoneally, into 2-month-old BALB/c mice, alone and combined with free or liposomal (in separate vesicles) recombinant human interleukin-2 (IL-2). Liposomes (MLV) consisted of DMPC/DMPG (9/1 mole ratio) were prepared by the POST technique described above at a lipid/HN and lipid/IL-2 w/w ratio of 300/1. The antigen dose was 0.25 μg HN of each viral strain and the IL-2 dose was 3.3 μg (60,000 IU).

The humoral response was tested on days 15 and 30 post-vaccination using the hemagglutination inhibition (I) assay.

As can be seen in Table 6, co-administration of liposomal IL-2 as an adjuvant (group 4) induced a significantly greater response, determined by HI titer and % seroconversion, than free IL-2 (groups) against all 3 strains and at both time points. Similar results were obtained in aged mice (18-months-old) (data not shown).

TABLE 6
The anti-hemagglutinin response of BALB/c mice vaccinated with a free/liposomal trivalent
subunit influenza vaccine, alone and combined with free/liposomal IL-2

| | HI titer (mean ± SD) against^{a} | | | | | |
|---|---|---|---|---|---|---|
| Vaccine | A/Sydney | | A/Beijing | | B/Yamanasbi | |
| (n = 5/group) | Day 15 | Day 30 | Day 15 | Day 30 | Day 15 | Day 30 |
| 1. Free HN | 12 ± 16 (20) | 32 ± 39 (40) | 0 (0) | 5 ± 8 (0) | 0 (0) | 0 (0) |
| 2. Lip-HN | 15 ± 16 (20) | 320 ± 160 (60) | 0 (0) | 24 ± 32 (40) | 4 ± 8 (0) | 8 ± 16 (20) |
| 3. Lip-HN + free IL-2 | 48 ± 16 (80) | 512 ± 256 (80) | 32 ± 16 (80) | 224 ± 74 (100) | 12 ± 10 (20) | 48 ± 30 (80) |
| 4. Lip-HN + Lip-IL-2 | 160 ± 87 (100) | 640 ± 0 (100) | 160 ± 0 (100) | 640 ± 0 (100) | 32 ± 10 (60) | 144 ± 32 (100) |

^{a}The values in parentheses indicate the % seroconversion (% of mice with an HI titer ≧ 40). The HI titers of group 4 are significantly greater (P < 0.05, Student t test), as compared with all other groups

Effect of Storage of Freeze-Dried Lipids on Human IL-2 Encapsulation

IL-2-loaded MLVs were prepared by the POST technique as described above, using DMPC/DMPG (9/1 mole ratio) that were dissolved in tertiary butanol, freeze-dried overnight, and stored at 4° C. for 20 months prior to hydration with the IL-2 solution. The encapsulation efficiency (˜80%), the mean liposomal size (˜1.5 μm), and stability (≦10% IL-2 leakage after 3 months at 4° C.) were similar to those of liposomal IL-2 prepared with freshly freeze-dried lipids.

### Example 6

### Efficacy of a Combined Liposomal Influenza Vaccine in Human Volunteers

Based on the successful pre-clinical studies in mice, which showed enhanced immune response following vaccination with a combined vaccine consisting of liposomal influenza antigens (HN) and liposomal IL-2 (see Table 6) and a good safety profile in rabbits, the combined vaccine (designated INFLUSOME-VAC) was tested in 2 clinical trials in 2000/2001. One trial was conducted in healthy young adults (mean age 28 y., n=53) and the second in nursing-home residents (mean age 81 y., n=81). The volunteers were randomized to receive a single intramuscular administration of either the standard (commercial) trivalent vaccine (15 μg of each viral strain, subunit or split viron preparation) or INFLUSOME-VAC that was prepared from the same vaccine. The combined liposomal vaccine comprised of DNPC/DMPG (9/1 mole ratio) liposomes loaded with the influenza antigens and with rhIL-2 (600,000 IU/dose), in separate liposomes. The liposomes were prepared by the POST encapsulation technique (the present invention), using an approximately 500/1 lipid/protein w/w ratio, for HN and IL-2.

Results and Conclusions

The response was tested prior to and 28 days post-vaccination using the hemagglutination-inhibition (HI) assay. As can be seen in Table 7, INFLUSOME-VAC was significantly more efficient (P <0.05, Fisher exact test) against the three viral strains in the young volunteers and against the two A strains in the elderly, as determined by % seroconversion (% of vaccines with a ≧4fold increase in HI titer, achieving a titer of 2:40 on day 28). No increase in adverse reactions (except for local pain in the young volunteers) was observed in either study. Thus, INFLUSOME-VAC is both safe and more immunogenic than the standard influenza vaccine in young volunteers and the elderly.

TABLE 7
The anti-hemagglutinin response (HI) of human volunteers
vaccinated with standard influenza vaccine or INFLUSOME-VAC

| | | % Seroconversion against: | | |
|---|---|---|---|---|
| | | | | B/ |
| Trial | Vaccine | A/Sydney | A/Beijing | Yamanashi |
| Young | Standard (n = 17) | 35 | 65 | 35 |
| volunteers | INFLUSOME-VAC | 69^{a} | 97^{a} | 69^{a} |
| | (n = 36) | | | |
| | | A/New | | B/ |
| | | Caledonia | A/Moscow | Yamanashi |
| Elderly | Standard (n = 33) | 45 | 24 | 9 |
| volunteers | INFLUSOME-VAC | 65^{a} | 44^{a} | 19 |
| | (n = 48) | | | |

^{a}P < 0.05 (Fisher exact test) compared with the standard vaccine.

1-53. (canceled)

54. A method for loading biological material in liposomal vesicles comprising:
i) solubilizing at least one liposome-forming lipid in a solvent and freeze-drying the same to effect a dry liposome-forming lipid;
ii) providing an aqueous solution of biological material;
iii) hydrating the freeze-dried liposome-forming lipid with the solution of the biological material in a manner to effect loading of said biological material in liposomes formed from the liposome-forming lipid.

55. The method of claim 54, wherein said liposome-forming lipid is selected from phospholipids, lipopolymers, cationic lipids, sphingolipids a combination thereof and a combination thereof with membrane active sterols.

56. The method of claim 56, wherein said phospholipids is selected from hydrogenated, partially hydrogenated or non-hydrogenated phospholipids, all derived from a natural source, said natural source is selected from egg, yolk, milk, rice or soybeans.

57. The method of claim 54, wherein said phospholipids are fully synthetic or semi-synthetic phospholipids selected from dimyristoyl phosphatidylcholine (DMPC), dimyristoyl phosphatidylglycerols (DMPG), phosphatidylglycerols, phosphatidylinositols, phosphatidylserines, sphingomyelins, or mixture thereof

58. The method of claim 57, wherein said phospholipids comprise a mixture of DMPC and DMPG.

59. The method of claim 58, wherein said mixture of DMPC and DMPG is at a molar ratio of between 1:20 and 20:1

60. The method of claim 55, wherein said lipopolymers are PEGylated lipids.

61. The method of claim 55, wherein said sphingolipids are sphingomyelins (SPM) selected from egg-derived SPM, milk-derived SPM, N-palmitoyl-SPM, N-stearoyl-SPM, N-oleoyl-SPM (C18:1), N-nervacyl C (C24:1) SPM, N-lignoceryl SPM (C24:0), or a mixture thereof.

62. The method of claim 55, wherein said cationic lipids are monocationic lipids selected from 1,2-dimyristoyl-3-trimethylammonium propane (DMTAP), 1,2-dioleoyl-3-trimethylammonium propane (DOTAP), 1,2-distearoyl-3-trimethylammonium propane (DSTAP), or a polycationic lipid being spermine-based N-[2-[[2,5-bis[(3-aminopropyl)amino]-1-oxopentyl]amino]ethyl]-N,N-dimethyl-2,3-bis[(1-oxo-9-octadecenyl)oxy]-1-propanaminium 1,2-dimyristoyl-3-trimethylammonium propane (DOSPA), or a cationic lipid modified with cholesterol.

63. The method of claim 54, wherein said biological material comprises biological cell structures, cell products, and natural or synthetic biopolymers and/or oligomers.

64. The method of claim 63, wherein said biological cell structures comprise cell membranes, ribosomes, or mitochondriae; and said biopolymers or oligomers are enzymes, proenzymes, cofactors, receptors, virions, or virion surface antigens, bacteria or other pathogens, their membranes, fragments and surface antigens; antigens, antibodies, complement factors, hormones, cytokines, growth factors, nucleotides, DNA, mRNA, rRNA, tRNA, iRNA, antisense DNA or antisense RNA.

65. The method of claim 64, wherein said biological material is an immunoadjuvant.

66. The method of claim 65, wherein said immunoadjuvant is an immunostimulatory oligodeoxynucleotide sequence (ISS-ODN).

67. The method of claim 64, wherein said biological material is an antigen or a mixture of antigens.

68. The method of claim 64, wherein said biological material is a peptide or peptide mixture.

69. The method of claim 64, wherein said biological material is an antisense oligonucleotide.

70. The method of claim 64, wherein said biological material is a cytokine.

71. The method of claim 64, wherein said biological material is a combination of an immunoadjuvant and at least one antigen.

72. The method of claim 54 wherein said solvent is a polar, water miscible solvent or an apolar solvent.

73. The method of claim 72, wherein said polar, water miscible solvent is tertiary-butanol.

74. The method of claim 72, wherein said apolar solvent is cyclohexane.

75. The method of claim 54, wherein said solution of biological material is a solution thereof in sterile water or in a physiologically acceptable aqueous solution selected from the group consisting of 0.9% NaCl, buffered Saline, 5% dextrose, buffered dextrose, 10% sucrose and buffered sucrose.

76. The method of claim 54 for achieving more than 60% loading of biological material in liposomes.

77. A pharmaceutical formulation comprising as active ingredient a therapeutically effective amount of liposomes loaded with a biological material and a pharmaceutically acceptable additive, the loaded liposomes being prepared by the method of claim 54.

78. The pharmaceutical formation of claim 77, wherein said liposomes are formed from liposome-forming lipids, the liposome forming lipids being selected from phospholipids, lipopolymers, cationic lipids, sphingolipids a combination thereof and a combination thereof with membrane active sterols.

79. The pharmaceutical formation of claim 78, wherein said phospholipids is selected from hydrogenated, partially hydrogenated or non-hydrogenated phospholipids, all derived from a natural source, said natural source is selected from egg, yolk, milk, rice or soybeans.

80. The pharmaceutical formation of claim 78, wherein said phospholipids are fully synthetic or semi-synthetic phospholipids selected from dimyristoyl phosphatidylcholine (DMPC), dimyristoyl phosphatidylglycerols (DMPG), phosphatidylglycerols, phosphatidylinositols, phosphatidylserines, sphingomyelins, or mixture thereof.

81. The pharmaceutical formation of claim 78, wherein said phospholipids comprise a mixture of DMPC and DMPG.

82. The pharmaceutical formation of claim 81, wherein said mixture of DMPC and DMPG is at a molar ratio of between 1:20 and 20:1

83. The pharmaceutical formation of claim 78, wherein said lipopolymers are PEGylated lipids.

84. The pharmaceutical formation of claim 78, wherein said sphingolipids are sphingomyelins (SPM) selected from egg-derived SPM, milk-derived SPM, N-palmitoyl-SPM, N-stearoyl-SPM, N-oleoyl-SPM (C18:1), N-nervacyl C (C24:1) SPM, N-lignoceryl SPM (C24:0), or a mixture thereof.

85. The pharmaceutical formation of claim 78, wherein said cationic lipids are monocationic lipids selected from 1,2-dimyristoyl-3-trimethylammonium propane (DMTAP), 1,2-dioleoyl-3-trimethylammonium propane (DOTAP), 1,2-distearoyl-3-trimethylammonium propane (DSTAP), or a polycationic lipid being spermine-based N-[2-[[2,5-bis[(3-aminopropyl)amino]-1-oxopentyl]amino]ethyl]-N,N-dimethyl-2,3-bis[(1-oxo-9-octadecenyl)oxy]-1-propanaminium 1,2-dimyristoyl-3-trimethylammonium propane (DOSPA), or a cationic lipid modified with cholesterol.

86. The pharmaceutical formation of claim 77, wherein said biological material comprises biological cell structures, cell products, and natural or synthetic biopolymers and/or oligomers.

87. The pharmaceutical formation of claim 86, wherein said biological cell structures comprise cell membranes, ribosomes, or mitochondriae; and said biopolymers or oligomers are enzymes, proenzymes, cofactors, receptors, virions, or virion surface antigens, bacteria or other pathogens, their membranes, fragments and surface antigens; antigens, antibodies, complement factors, hormones, cytokines, growth factors, nucleotides, DNA, mRNA, rRNA, tRNA, iRNA, antisense DNA or antisense RNA.

88. The pharmaceutical formation of claim 86, wherein said biological material is an immunoadjuvant.

89. The pharmaceutical formation of claim 88, wherein said immunoadjuvant is an immunostimulatory oligodeoxynucleotide sequence (ISS-ODN).

90. The pharmaceutical formation of claim 87, wherein said biological material is an antigen or a mixture of antigens.

91. The pharmaceutical formation of claim 87, wherein said biological material is a peptide or peptide mixture.

92. The pharmaceutical formation of claim 87, wherein said biological material is an antisense oligonucleotide.

93. The pharmaceutical formation of claim 87, wherein said biological material is a cytokine.

94. The pharmaceutical formation of claim 88, wherein said biological material is a combination of an immunoadjuvant and at least one antigen.

95. The pharmaceutical formation of claim 77, comprising more than 60% of the biological material loaded in said liposomes.

96. A method for the prevention or treatment of a disease comprising administering to a subject in need a therapeutically effective amount of a pharmaceutical formulation according to claim 78.

97. The method of claim 96, wherein said liposomes are formed from a mixture of DMPC and DMPG.

98. The method of claim 97, wherein said mixture of DMPC and DMPG is at a molar ratio of between 1:20 and 20:1.

99. The method of claim 96, wherein said liposomes are formed PEGylated lipids.

100. The method of claim 96, wherein said biological material is an immunoadjuvant.

101. The method of claim 96, wherein said immunoadjuvant is an immunostimulatory oligodeoxynucleotide sequence (ISS-ODN).

102. The method of claim 96, comprising administration of said pharmaceutical formulation in combination with at least one antigen, said antigen being in a free form, or encapsulated in a liposome.

103. The method of claim 96, wherein said pharmaceutical formulation comprises at least 60% of said biological material loaded onto liposomes.

104. The method of claim 96, wherein said effective amount is a dosage of up to 2,000 mg of loaded liposomal vesicles, measured by phospholipid per kg body wt.
